(19) United States
(12) Patent Application Publication
(10) Pub. No.: US 2026/0209721 A1
(43) Pub. Date: Jul. 23, 2026

(54) GM3 SYNTHASE VECTORS AND USES THEREOF

(71) Applicants: University of Massachusetts, Westborough, MA (US); The Clinic for Special Children, Inc., Strasburg, PA (US)

(72) Inventors: Guangping Gao, Worcester, MA (US); Huiya Yang, Worcester, MA (US); Kevin A. Strauss, Strasburg, PA (US); Dan Wang, Worcester, MA (US)

(73) Assignees: University of Massachusetts, Westborough, MA (US); The Clinic for Special Children, Inc., Strasburg, PA (US)

(21) Appl. No.: 19/446,200

(22) Filed: Jan. 12, 2026

Related U.S. Application Data
(63) Continuation of application No. 17/602,348, filed on Apr. 10, 2020, now Pat. No. 12,553,036.
(60) Provisional application No. 62/833,136, filed on Apr. 12, 2019.

(51) Int. Cl. C12N 9/10; A61K 48/00; C12N 15/86

(52) U.S. Cl. CPC C12N 9/1081; A61K 48/0058; A61K 48/0075; C12N 15/86; C12Y 204/99009; C12N 2750/14143; C12N 2800/22; C12N 2800/90; C12N 2830/007; C12N 2830/50; C12N 2840/102

(57) ABSTRACT

Aspects of the disclosure relate to compositions and methods for expressing one or more Ganglioside GM3 synthase (GM3S) isoforms in a cell or subject. In some aspects, the disclosure relates to methods for treating GM3 synthase deficiency in a subject in need thereof.

### RELATED APPLICATIONS

This application is a continuation of U.S. application Ser. No. 17/602,348, filed Oct. 8, 2021, which is a national stage filing under 35 U.S.C. 371 of international PCT application PCT/US2020/027585, filed Apr. 10, 2020, which claims priority under 35 USC § 119 (e)) to U.S. provisional patent application 62/833,136, filed Apr. 12, 2019, the entire contents of each of which are incorporated by reference herein.

### REFERENCE TO AN ELECTRONIC SEQUENCE LISTING

The contents of the electronic sequence listing (U012070113US02-SEQ-KZM.xml; Size: 238,859 bytes; and Date of Creation: Jan. 12, 2026) are herein incorporated by reference in their entirety.

### BACKGROUND

GM3 synthase (GM3S) deficiency is a rare monogenic neurological disorder common within Old Order Amish communities. In some cases, GM3S deficiency is associated with an ST3GAL5 c.862C>T founder variant segregating with a population-specific carrier frequency of ~4%. GM3S mediates synthesis of GM3, which serves as the common precursor for all cerebral gangliosides. GM3S deficiency abolishes ganglioside biosynthesis. ST3GAL5 c.862C>T homozygotes appear healthy at birth, but develop progressive microcephaly, neurodevelopmental stagnation, intractable epilepsy, irritability, insomnia, deafness, blindness, and dyskinesia within a few months of life. No treatment is currently available.

### SUMMARY

Aspects of the disclosure relate to compositions and methods for expressing GM3 synthase (GM3S) proteins in a subject. The disclosure is based, in part, on expression constructs encoding one or more GM3S isoforms (e.g., one or more Ia Type 1 isoforms, Ia Type 2 isoforms, 1b Type 1 isoforms, Ib Type 2 isoforms, Ic isoforms, and combinations thereof). In some embodiments, expression constructs described by the disclosure are useful for treating diseases associated with GM3S deficiency.

Accordingly, in some aspects, the disclosure provides an isolated nucleic acid comprising: (i) a first region comprising a first adeno-associated virus (AAV) inverted terminal repeat (ITR); and (ii) a second region comprising a transgene having a nucleic acid sequence encoding one or more monosialodihexosylganglioside synthase (GM3S) protein isoforms.

In some aspects, the disclosure provides an isolated nucleic acid comprising: (i) a first region comprising a first adeno-associated virus (AAV) inverted terminal repeat (ITR); and (ii) a second region comprising a transgene having a nucleic acid sequence encoding one or more monosialodihexosylganglioside synthase (GM3S) Ia Type 2 protein isoforms. In some embodiments, the GM3S Ia Type 2 protein isoforms are encoded by the nucleic acid sequences set forth in SEQ ID NO: 40-42.

In some embodiments, a transgene encodes a one or more GM3S Ia isoforms. In some embodiments, one or more GM3S isoforms are GM3S Ia Type 1 isoforms. In some embodiments, one or more GM3S Ia Type 1 isoforms are translated from an M2 initiation codon. In some embodiments, GM3S Ia Type 1 isoform translated from an M2 initiation codon are encoded by the nucleic acid sequence set forth in SEQ ID NO: 41. In some embodiments, one or more GM3S Ia Type 1 isoforms are translated from an M3 initiation codon. In some embodiments, GM3S Ia Type 1 isoform translated from an M3 initiation codon are encoded by the nucleic acid sequence set forth in SEQ ID NO: 42.

In some embodiments, one or more GM3S isoforms are GM3S Ia Type 2 isoforms. In some embodiments, one or more GM3S Ia Type 2 isoforms are translated from an M1 initiation codon. In some embodiments, GM3S Ia Type 2 isoform translated from an M1 initiation codon are encoded by the nucleic acid sequence set forth in SEQ ID NO: 39 or 40. In some embodiments, one or more GM3S Ia Type 2 isoforms are translated from an M2 initiation codon. In some embodiments, GM3S Ia Type 2 isoform translated from an M2 initiation codon are encoded by the nucleic acid sequence set forth in SEQ ID NO: 41. In some embodiments, one or more GM3S Ia Type 2 isoforms are translated from an M3 initiation codon. In some embodiments, GM3S Ia Type 2 isoform translated from an M3 initiation codon are encoded by the nucleic acid sequence set forth in SEQ ID NO: 42.

In some embodiments, a transgene encodes a one or more GM3S Ib isoforms. In some embodiments, one or more GM3S isoforms are GM3S Ib Type 1 isoforms. In some embodiments, one or more GM3S Ib Type 1 isoforms are translated from an M2 initiation codon. In some embodiments, GM3S Ib Type 1 isoform translated from an M2 initiation codon are encoded by the nucleic acid sequence set forth in SEQ ID NO: 41. In some embodiments, one or more GM3S Ib Type 1 isoforms are translated from an M3 initiation codon. In some embodiments, GM3S Ib Type 1 isoform translated from an M3 initiation codon are encoded by the nucleic acid sequence set forth in SEQ ID NO: 42.

In some embodiments, one or more GM3S isoforms are GM3S Ib Type 2 isoforms. In some embodiments, one or more GM3S Ib Type 2 isoforms are translated from an M2 initiation codon. In some embodiments, GM3S Ib Type 2 isoform translated from an M2 initiation codon are encoded by the nucleic acid sequence set forth in SEQ ID NO: 41. In some embodiments, one or more GM3S Ib Type 2 isoforms are translated from an M3 initiation codon. In some embodiments, GM3S Ib Type 2 isoform translated from an M3 initiation codon are encoded by the nucleic acid sequence set forth in SEQ ID NO: 42.

In some embodiments, a transgene encodes a one or more GM3S Ic isoforms. In some embodiments, one or more GM3S isoforms are translated from an M2′ initiation codon. In some embodiments, one or more GM3S isoforms are translated from an M2 initiation codon. In some embodiments, GM3S Ic isoform translated from an M2 initiation codon are encoded by the nucleic acid sequence set forth in SEQ ID NO: 41. In some embodiments, one or more GM3S isoforms are translated from an M3 initiation codon. In some embodiments, GM3S Ic isoform translated from an M3 initiation codon are encoded by the nucleic acid sequence set forth in SEQ ID NO: 42.

In some embodiments, a transgene further comprises a 5′ untranslated region (5′UTR), such as a ST3GAL5 5′UTR. In some embodiments, a transgene does not include a 5′UTR, such as a ST3GAL5 5′UTR.

In some embodiments, a transgene further comprises a Kozak sequence (GCCACC) operably linked to the nucleic acid sequence encoding the one or more GM3S protein isoforms. In some embodiments, a transgene does not comprise a Kozak sequence (GCCACC).

In some embodiments, each of the one or more GM3S protein isoforms comprises or consists of the amino acid sequence set forth in any one of SEQ ID NOs: 8-10.

In some embodiments, a transgene further comprises a promoter operably linked to the nucleic acid sequence encoding the one or more GM3 synthase protein isoforms. In some embodiments, a promoter is a chicken beta-actin (CBA) promoter. In some embodiments, a promoter (e.g., a CBA promoter) comprises a CMV enhancer sequence.

In some embodiments, a transgene further comprises a third region comprising a second adeno-associated virus (AAV) inverted terminal repeat (ITR), or a variant thereof. In some embodiments, a transgene is flanked by AAV ITRs. In some embodiments, an AAV ITR is an AAV2 ITR or a variant thereof.

In some embodiments, a nucleic acid sequence encoding one or more GM3S protein isoforms is codon-optimized.

In some aspects, the disclosure provides an isolated nucleic acid comprising the sequence set forth in any one of SEQ ID NOs: 1-7, and 11-38.

In some embodiments, an isolated nucleic acid described by the disclosure comprises a one or more miRNA binding sites (e.g., a fourth region comprising one or more miRNA binding sites). In some embodiments, one or more miRNA binding sites are miR-122 binding sites. In some embodiments, one or more miRNA binding sites are positioned between the last codon of a nucleic acid sequence encoding one or more GM3S protein isoforms and a polyA tail region.

In some aspects, the disclosure provides a vector comprising an isolated nucleic acid as described herein. In some embodiments, the vector is a plasmid. In some embodiments, the vector is a viral vector. In some embodiments, the viral vector is an adenoviral vector, adeno-associated virus (AAV) vector, lentiviral vector, retroviral vector, or Baculovirus vector.

In some aspects, the disclosure provides a host cell comprising an isolated nucleic acid or a vector as described herein.

In some aspects, the disclosure provides a recombinant adeno-associated virus (rAAV) comprising: (i) a capsid protein; and (ii) an isolated nucleic acid as described herein. In some embodiments, a capsid protein is an AAV9 capsid protein or a variant thereof. In some embodiments, an AAV9 capsid protein variant is AAV.PHP.B capsid protein or AAB.PHP.eB capsid protein.

In some embodiments, an rAAV is formulated for delivery to the brain.

In some aspects, the disclosure provides a composition comprising an rAAV as described herein, and a pharmaceutical acceptable excipient.

In some aspects, the disclosure provides a method for expressing one or more GM3S protein isoforms in a cell, the method comprising delivering to the cell an isolated nucleic acid, vector, or rAAV as described herein.

In some aspects, the disclosure provides a method for treating a GM3 synthase (GM3S) deficiency in a subject in need thereof, the method comprising administering to a subject having a GM3 synthase (GM3S) deficiency an effective amount of an isolated nucleic acid, vector, or rAAV as described herein.

In some embodiments, a subject is a human. In some embodiments, a subject is characterized as having one or more mutations in a ST3GAL5 gene. In some embodiments, one or more mutations occurs at position c.862. In some embodiments, the mutation is C862T of SEQ ID NO: 39. In some embodiments, one or more mutations occurs at position c. 1063. In some embodiments, the mutation is G1063A of SEQ ID NO: 39. In some embodiments, one or more mutations occurs at positions c.584 and c.601. In some embodiments, the mutations are at positions c.G584C of SEQ ID NO: 39 and c.G601A of SEQ ID NO: 39.

In some embodiments, the administration results in delivery of an isolated nucleic acid or rAAV to the brain of the subject. In some embodiments, administration occurs via systemic injection or direct injection to the central nervous system (CNS). In some embodiments, systemic injection is intravenous injection. In some embodiments, direct injection to the central nervous system (CNS) is intracerebral administration, intrathecal administration, or a combination thereof.

### BRIEF DESCRIPTION OF DRAWINGS

FIGS. 1A-1C show human ST3GAL5 protein isoforms and constructs expressing hST3GAL5 and their expression in Hela cells. FIG. 1A shows a schematic of a hST3GAL5-1a-2 RNA variant has at least three protein products that initiate from different start codons. FIG. 1B shows seven AAV constructs expressing the human ST3GAL5 cDNA, driven by the CMV/CB promoter. FIG. 1C shows a Western blot showing hST3GAL5 expression following transfection in Hela cells with the corresponding plasmids. All the plasmids can produce the desired isoforms and the 5′-UTR decreases the protein expression in Hela cells.

FIGS. 2A-2B show constructs expressing hST3GAL5 protein in patient-derived fibroblast cells. FIG. 2A shows patient-derived fibroblast cells genotype information. FIG. 2B shows a Western blot showing hST3GAL5 expression following transfection in fibroblast cells with the corresponding plasmids. All the plasmids produce the desired isoforms.

FIGS. 3A-3B show quantification of in vivo rAAV genome delivery and transgene expression of wild-type mice by droplet digital PCR (ddPCR). FIG. 3A shows rAAV genome delivery data. FIG. 3B shows transgene expression data. Each dot represents the tissue from one animal (n=3~5).

FIGS. 4A-4B show Liquid chromatography-Tandem mass spectrometry (LC-MS/MS) analysis of ganglioside standards using Hydrophilic interaction liquid chromatography (HILIC) column chemistry. FIG. 4A shows 18:1 subspecies of ganglioside standards. FIG. 4B shows 20:1 subspecies of ganglioside standards. From top to bottom: GM1, GM2, GM3, GD1b, GD2, and GD3

FIG. 5 shows anti-GM3 immunofluorescence analysis in transfected fibroblast cells. FIG. 5 shows healthy control derived fibroblast cells. ST3GAL5+/M is the healthy control line. ST3GAL5 M/M is generated from patient. ST3GAL5 M/M either remains untreated, or treated with lentivirus

FIG. 6 shows a schematic of constructs expressing hST3GAL5 and miR-122 binding sites. Four AAV constructs expressing the human ST3GAL5 cDNA, driven by the CMV/CB promoter are shown. Three miR-122 binding sites are added at the end of the transgene.

FIGS. 7A-7B show patient iPSC-induced cortical neurons. FIG. 7A shows a flow chart showing the cortical neuron generation timeline. FIG. 7B shows immunofluorscence staining showing the cortical neuron markers from D44 induced cortical neurons.

FIG. 8 shows anti-brain gangliosides analysis in iPSC-induced cortical neurons. The figure includes DAPI stain for nucleus, and GD1a, GD1b, or GT1b staining from top row to bottom row. ST3GAL5+/+ is the healthy control line. ST3GAL5 M/M is generated from patient iPSC. ST3GAL5 M/M either remains untreated, or treated with lentivirus.

FIGS. 9A-9B are tables showing the animal survival post AAV9_hST3GAL5 vectors injection at multiple doses. FIG. 9A is a table showing neonatal facial vein injection of the rAAV listed therein. FIG. 9B is a table showing young adult tail vein injection of the rAAV listed therein.

FIG. 10 shows the ALT level of wildtype young adult C57/BL6 mice post AAV9_hST3GAL5 vector I.V. injection. Blood are taken from animals weekly. Each dot represents the tissue from one animal (n=3~5).

FIG. 11 shows examples of constructs expressing hST3GAL5 under different expression cassettes. Cartoons showing four AAV expression cassettes expressing the human ST3GAL5 cDNA, driven by hSyn1 promoter, hST3GAL5 promoter and miR122 binding sites, hST3GAL5 endogenous full-length promoter, and hST3GAL5 endogenous truncated promoter.

FIGS. 12A-12C show quantification of in vivo rAAV genome delivery and transgene expression of wild-type mice by droplet digital PCR (ddPCR) and Western blot. FIG. 12A shows rAAV genome delivery by ddPCR. FIGS. 12B-12C show transgene expression by ddPCR and Western blot. Each dot represents the tissue from one animal (n=3~5).

### DETAILED DESCRIPTION

In some aspects, the disclosure relates to compositions and methods for expressing a transgene encoding one or more GM3 synthase (GM3S) proteins in a cell or subject. In some embodiments, the transgene encodes an isolated nucleic acid. In some embodiments, the isolated nucleic acid is comprised in a recombinant adeno-associated virus (rAAV).

Methods and compositions described by the disclosure may be utilized, in some embodiments, to treat diseases and disorders associated with GM3S deficiency.

### Isolated Nucleic Acids

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

Aspects of the disclosure relate to isolated nucleic acids encoding one or more GM3S proteins. GM3 synthase (GM3S), also referred to as Lactosylceramide alpha-2,3-sialyltransferase and SATI, is an enzyme that catalyzes formation of ganglioside GM3 using a lactosylceramide substrate. In some embodiments, a human GM3S protein is encoded by an mRNA transcript having the sequence set forth in any one of NCBI Reference Sequence Accession Numbers: NM_001042437, NM_003896, NM_001354226, NM_001354227, and NM_001354233. In some embodiments, a human GM3S protein comprises the amino acid sequence set forth in any one of NCBI Reference Sequence Accession Numbers: NP_001035902, NP_003887, NP_001341155, NP_001341156, and NP_001341162.

Human GM3S transcripts (e.g., GM3S proteins translated from such transcripts) are generally classified into three isoforms, GM3S-Ia, -Ib, and -Ic, according to the position of transcription initiation, exon 1, 2, or 4. Additionally, GM3S-Ia and -Ib isoforms are each further classified into two types: GM3S-Ia Type 1 and Type 2 variants, and GM3S-Ib Type 1 and Type 2 variants, respectively, reflecting alternative splicing of exon 3. The structural features of GM3 isoforms are known, for example as described in Uemura et al. Mol Biol Cell. 20 (13): 3088-3100.

In some embodiments, an isolated nucleic acid includes an RNA transcript having one or more initiation codons (referred to as M1, M2, M3, etc.) which encodes one or more variants of a GM3S isoform (e.g., one or more variants of a Ia Type 1 isoform, one or more variants of a Ia Type 2 isoform, one or more variants of a Ib Type 1 isoform, one or more variants of a Ib Type 2 isoform, etc.). An initiation codon may be a codon encoding a methionine (M, encoded by AUG codon) or a codon encoding an amino acid that is not methionine but is capable of initiation of protein translation (e.g., leucine, encoded by CUG codon). In some embodiments, an initiation codon consists of the nucleotide sequence AUG.

The position of the one or more initiation codons within an isolated nucleic acid may vary. In some embodiments, an initiation codon is positioned at the N-terminus of a nucleic acid sequence encoding one or more GM3S proteins (e.g., the first three nucleotides of the nucleic acid encode an AUG codon). In some embodiments, an initiation codon is positioned between nucleotide bases and 50 nucleotide bases downstream from the N-terminus of a nucleic acid sequence encoding one or more GM3S proteins. In some embodiments, an initiation codon is positioned between 20 nucleotide bases and 100 nucleotide bases downstream from the N-terminus of a nucleic acid sequence encoding one or more GM3S proteins. In some embodiments, an initiation codon is positioned between 50 nucleotide bases and 500 nucleotide bases downstream from the N-terminus of a nucleic acid sequence encoding one or more GM3S proteins. In some embodiments, an initiation codon encodes a methionine (M) positioned between 1 amino acid and 30 amino acids downstream from the N-terminus of a nucleic acid sequence encoding one or more GM3S proteins. In some embodiments, an initiation codon encodes a methionine (M) positioned between 10 amino acid and 100 amino acids downstream from the N-terminus of a nucleic acid sequence encoding one or more GM3S proteins.

The disclosure is based, in part, on expression constructs encoding one or more GM3S isoforms or variants (e.g., one or more Ia Type 1 isoforms, Ia Type 2 isoforms, 1b Type 1 isoforms, Ib Type 2 isoforms, Ic isoforms, and combinations thereof). In some embodiments, an isolated nucleic acid encodes one or more GM3S Ia Type 1 variants (e.g., M1 isoform, M2 isoform, M3 isoform, or any combination of the foregoing.) In some embodiments, an isolated nucleic acid encodes one or more GM3S Ia Type 2 variants (e.g., M1 isoform, M2 isoform, M3 isoform, or any combination of the foregoing). In some embodiments, an isolated nucleic acid encodes one or more GM3S Ib Type 1 variants (e.g., M2 isoform, M3 isoform, or a combination thereof). In some embodiments, an isolated nucleic acid encodes one or more GM3S Ib Type 2 variants (e.g., M2 isoform, M3 isoform, or a combination thereof). In some embodiments, an isolated nucleic acid encodes one or more GM3S Ic Type 1 variants (e.g., M2′ isoform, M2 isoform, M3 isoform, or any combination of the foregoing).

In some aspects, the disclosure relates to isolated nucleic acids comprising or lacking certain regulatory sequences. In some embodiments, isolated nucleic acids and rAAVs described herein comprise (or lack) one or more of the following structural features (e.g., control or regulatory sequences): a 5′ untranslated region (5′UTR), a promoter, an intron, a Kozak sequence, one or more miRNA binding sites, a rabbit beta-globulin (RBG) poly A sequence, and a 3′ untranslated region (3′UTR). In some embodiments, one or more of the foregoing control sequences is operably linked to a nucleic acid sequence encoding one or more GM3S proteins.

The disclosure is based, in part, on isolated nucleic acids encoding one or more GM3S proteins (e.g., one or more GM3S isoforms or variants, for example GM3S Ia Type 2 M1, M2, and M3 variants, etc.) that lack a 5′ UTR (e.g., a ST5GAL5 5′ UTR) and/or lack a Kozak sequence (e.g., GCCACC). Without wishing to be bound by any particular theory, constructs lacking a 5′ UTR and/or a Kozak sequence express increased levels of multiple isoforms of GM3S proteins relative to expression constructs which include a 5′ UTR and/or a Kozak sequence.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame.

In some embodiments, a transgene comprises a nucleic acid sequence encoding one or more GM3S proteins operably linked to a promoter. A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

Generally, a promoter can be a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is an RNA pol III promoter, such as U6 or H1. In some embodiments, a promoter is an RNA pol II promoter.

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene (e.g., ST3GAL5 promoter or hSyn1 promoter) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some aspects, the disclosure relates to isolated nucleic acids comprising a transgene encoding one or more GM3S proteins, and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more GM3S proteins, and a poly A sequence.

In some embodiments, a transgene comprises one or more (e.g., 1, 2, 3, 4, 5, or more) miRNA binding sites that de-target expression of the one or more GM3S proteins from liver cells. For example, in some embodiments, a transgene comprises one or more miR-122 binding sites.

In some embodiments, a transgene comprises one or more (e.g., 1, 2, 3, 4, 5, or more) miRNA binding sites that de-target expression of the one or more GM3S proteins from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell. or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs.

Recombinant Adeno-Associated Viruses (rAAVs) and Other Vectors

Aspects of the disclosure relate to vectors comprising an isolated nucleic acid encoding one or more GM3S proteins. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., ocular tissues, neurons). The AAV capsid is an important element in determining these tissue-specific targeting capabilities (e.g., tissue tropism). Thus, an rAAV having a capsid appropriate for the tissue being targeted can be selected.

In some embodiments, rAAVs of the disclosure comprise a nucleotide sequence as set forth in any one of SEQ ID NOs: 1-7 and 11-38, or encode one or more GM3s proteins each having an amino acid sequence as set forth in any one of SEQ ID NOs: 8-10. In some embodiments, rAAVs of the disclosure comprise a nucleotide sequence that is 99% identical, 95% identical, 90% identical, 85% identical, 80% identical, 75% identical, 70% identical, 65% identical, 60% identical, 55% identical, or 50% identical to a nucleotide sequence as set forth in SEQ ID NOs: 1-7 and 11-38.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for CNS tissue (e.g., brain tissue, spinal tissue, etc.). In some embodiments, an AAV capsid protein targets neuronal cells. In some embodiments, an AAV capsid protein is capable of crossing the blood-brain barrier (BBB).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP.B, AAV.PHP.eB, and variants of any of the foregoing. In some embodiments, an AAV capsid protein is of a serotype derived from a non-human primate, for example AAVrh8 serotype.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence encoding a transgene (e.g., one or more GM3S proteins). A “host cell” refers to any cell that harbors, or is capable of harboring, a substance of interest. Often a host cell is a mammalian cell. In some embodiments, a host cell is a neuron. In some embodiments, a host cell is a photoreceptor cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron or a glial cell (e.g., astrocyte, oligodendrocyte, etc.).

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

### Methods

Methods for delivering a transgene (e.g., an isolated nucleic acid encoding one or more GM3S proteins) to a subject are provided by the disclosure. In some embodiments, a subject tis administered one or more (e.g., 1, 2, 3, 4, 5, 6, or more) rAAVs, each rAAV encoding a different transgene (e.g., each encoding a different GM3S protein). The methods typically involve administering to a subject an effective amount of an isolated nucleic acid encoding the transgene. In some embodiments, expression constructs described by the disclosure are useful for treating diseases associated by GM3S deficiency.

In some aspects, the disclosure provides a method for treating a GM3 synthase (GM3S) deficiency in a subject in need thereof, the method comprising administering to a subject having a GM3 synthase (GM3S) deficiency an effective amount of an isolated nucleic acid, vector, or rAAV as described herein. A subject may be any mammalian organism, for example a human, non-human primate, horse, pig, dog, cat rodent, etc. In some embodiments a subject is a human.

As used herein, “GM3 synthase deficiency” refers to a neurological disorder that is characterized by recurrent seizures (e.g., epileptic seizures, grand mal seizures, etc.) and cognitive defects (e.g., severe intellectual disability), insomnia, deafness, blindness, and dyskinesia. Typically, subjects having GM3S deficiency comprise one or more mutations in a ST3GAL5 gene. A mutation may be a point mutation, non-sense mutation, non-sense mutation, frameshift mutation, etc. Examples of mutations include mutations at position c.862 (e.g., c.C862T), position c.1063 (e.g., c.G1063A), and positions c.584 and c.601 (e.g., c.G584C and c.G601A).

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to increase the expression of GM3S proteins, to extend the lifespan of a subject, to improve in the subject one or more symptoms of disease (e.g., a symptom of GM3S deficiency), etc. The effective amount will depend on a variety of factors such as, for example, the species, age, weight, health of the subject, and the tissue to be targeted, and may thus vary among subject and tissue as described elsewhere in the disclosure.

As used herein, the term “treating” refers to the application or administration of a composition encoding one or more GM3S proteins to a subject, who has a disease associated with GM3S deficiency, a symptom of a disease associated with GM3S deficiency, or a predisposition toward a disease associated with GM3S deficiency (e.g., one or more mutations in the ST3GAL5 gene), with the purpose to cure, heal, alleviate, relieve, alter, remedy, ameliorate, improve, or affect the disorder, the symptom of the disease, or the predisposition toward a disease associated with GM3S deficiency.

Alleviating a disease associated with GM3S deficiency includes delaying the development or progression of the disease, or reducing disease severity. Alleviating the disease does not necessarily require curative results. As used therein, “delaying” the development of a disease (such as a disease associated with GM3S deficiency) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with GM3S deficiency includes initial onset and/or recurrence.

In some embodiments, administration occurs via systemic injection or direct injection to the central nervous system (CNS). In some embodiments, systemic injection is intravenous injection. In some embodiments, direct injection to the central nervous system (CNS) is intracerebral administration, intrathecal administration, or a combination thereof.

### Administration

The isolated nucleic acids and rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

Aspects of the instant disclosure relate to compositions comprising one or more recombinant AAVs, each rAAV comprising a capsid protein and a nucleic acid encoding a transgene, wherein the transgene comprises a nucleic acid sequence encoding one or more GM3S proteins. In some embodiments, each GM3S protein independently comprises or consists of the sequence set forth in any one of SEQ ID NOs: 8-10. In some embodiments, the nucleic acid further comprises AAV ITRs. In some embodiments, the rAAV comprises an rAAV vector represented by the sequence set forth in any one of SEQ ID NO: 1-7, and 11-38, or a portion thereof. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intracerebroventricular, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ~10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLE

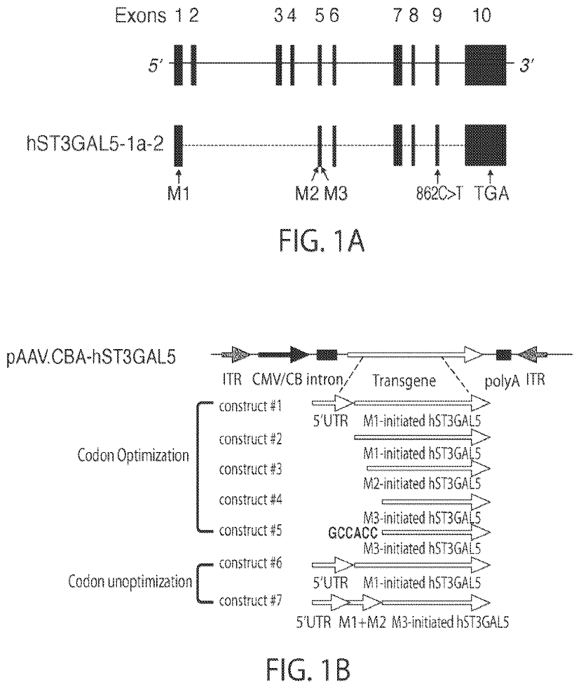

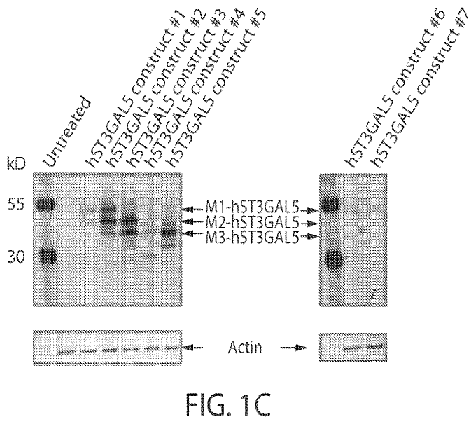

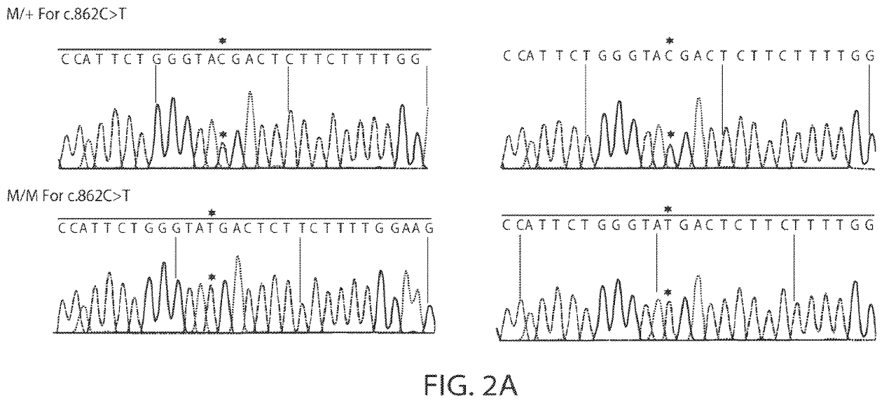

One GM3S mRNA variant (hST3GAL5-1a-2) has been observed to be the most abundant isoform in human tissues, including brain. However, hST3GAL5-1a-2 can produce at least three protein products that initiate from different start codons (FIG. 1A). Therefore, multiple hST3GAL5 constructs that differ in their initiating start codons, codon optimization, and the presence of the full length 5′-UTR (FIG. 1B) were produced.

Expression efficiency of the constructs was investigated by transfecting HeLa cells, which normally have no detectable endogenous ST3GAL5 expression. All constructs expressed the desired GM3S isoform. It was noted that constructs having a 5′-UTR exhibited reduced gene expression (FIG. 1C). Constructs without the 5′-UTR were investigated further.

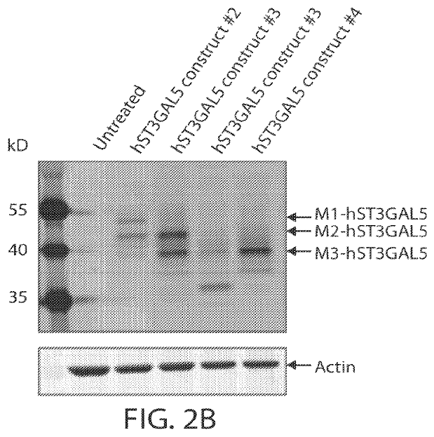

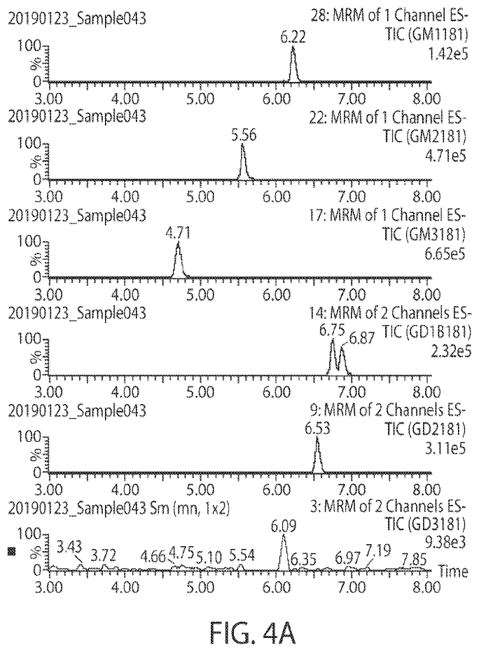

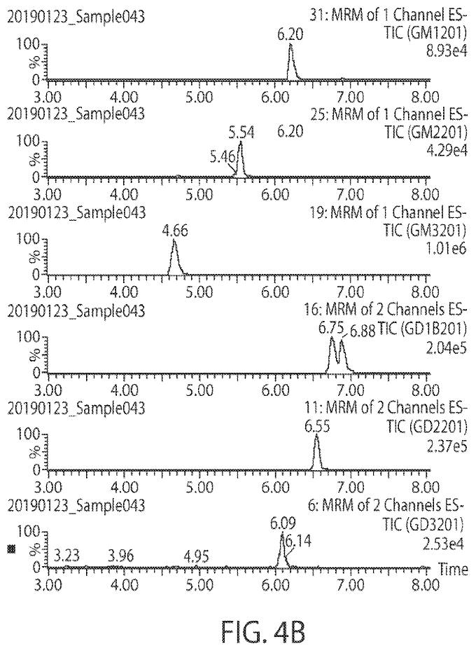

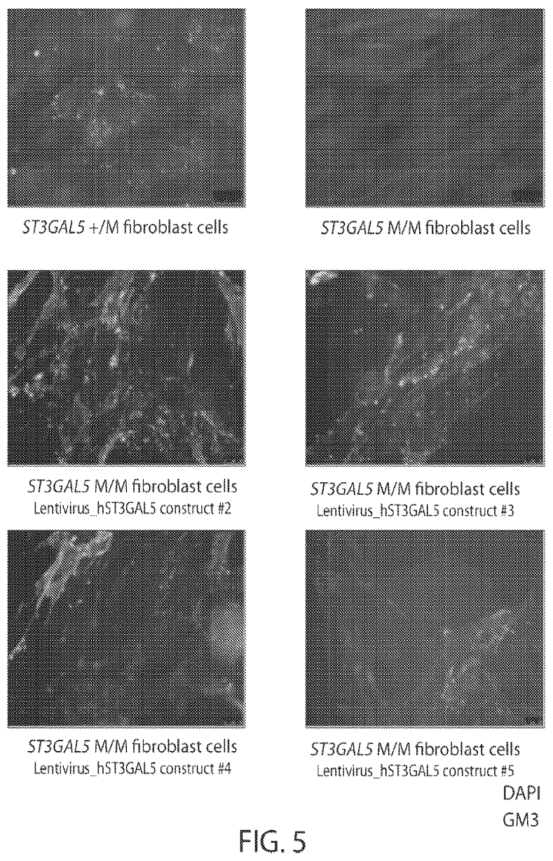

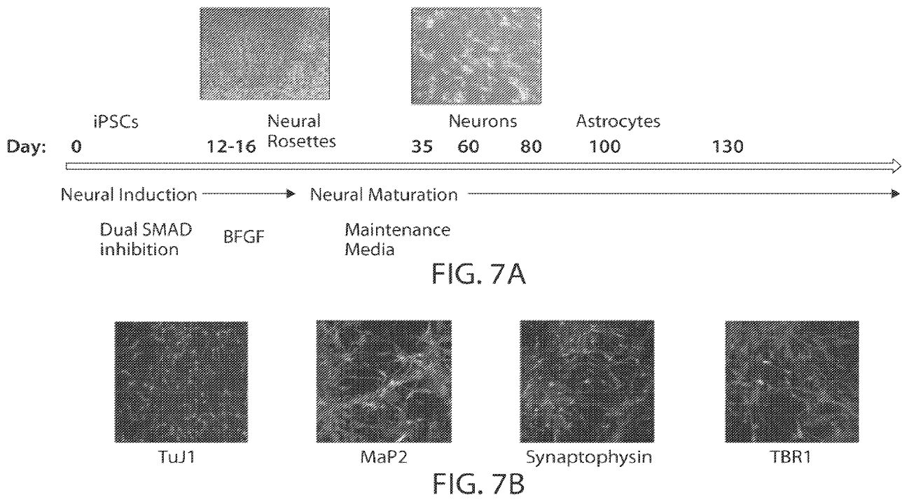

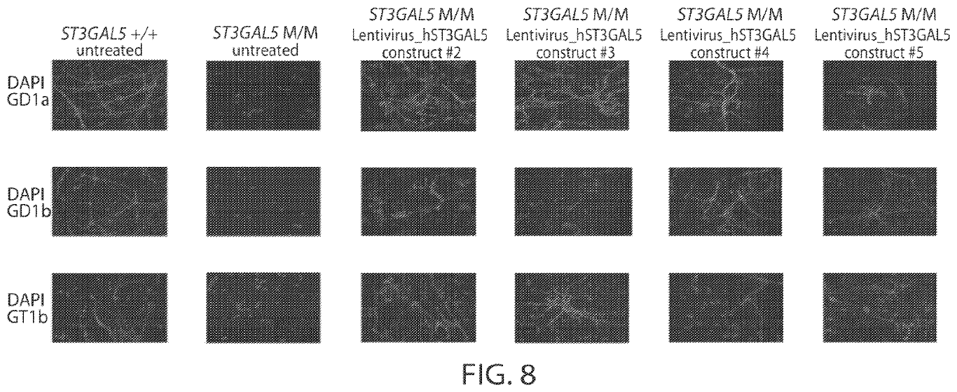

Transfection with ST3GAL5 constructs restored GM3S production in patient-derived fibroblasts (FIG. 2). Anti-GM3 antibody assays were performed to assess GM3 expression in patient-derived fibroblasts (FIG. 5). In addition, human ST3GAL5 cDNAs encoding each isoform of the ST3GAL5 protein were delivered into iPSC-derived cortical neurons from patients with ST3GAL5 deficiency by lentiviral-mediated gene delivery (FIGS. 7A-7B). hST3GAL5 gene replacement in iPSC-derived cortical neurons reconstituted GM3 as well as its major downstream a- and b-series brain gangliosides (e.g., GD1a, GD1b, and GT1b) (FIG. 8). These in vitro data demonstrates the strong therapeutic potential of human ST3GAL5 cDNA gene replacement therapy. Liquid chromatography-tandem mass spectrometry (LC-MS/MS) technique is used to analyze the downstream gangliosides biosynthesis. Example LC-MS/MS data is shown in FIGS. 4A-4B.

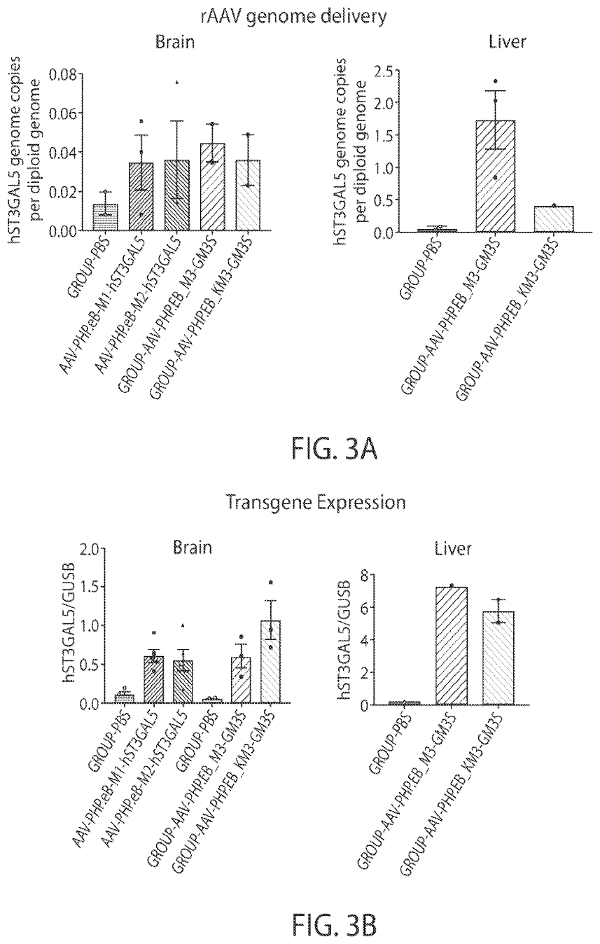

The hST3GAL5 constructs were packaged into AAV9 or AAV-PHP.eB capsids that efficiently target murine neurons and astrocytes. To examine the safety and efficacy of transgene expression, AAV vectors were injected via facial vein into neonatal wild type mice. There was no observed dose-related toxicity. AAV vector delivery and GM3S protein over-expression in the brain were quantified by Droplet Digital PCR (ddPCR) and western blot, respectively (FIGS. 3A-3B), and no short-term vector-associated toxicity (FIG. 13) was observed.

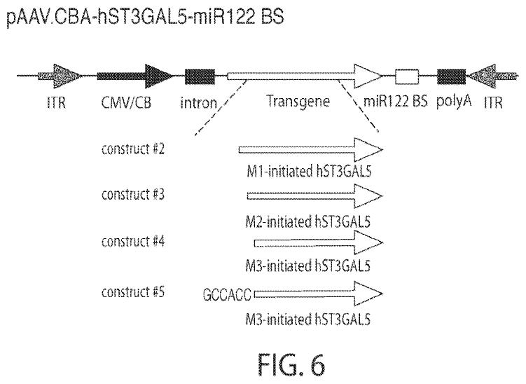

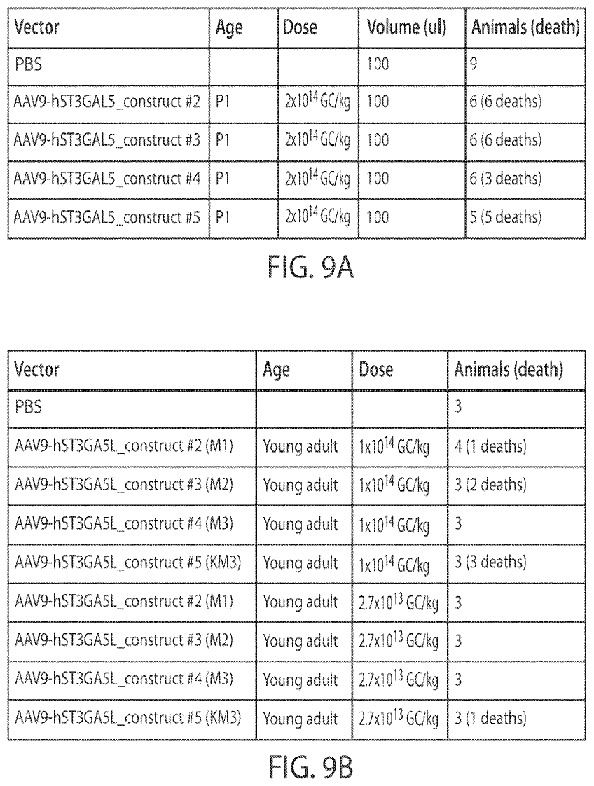

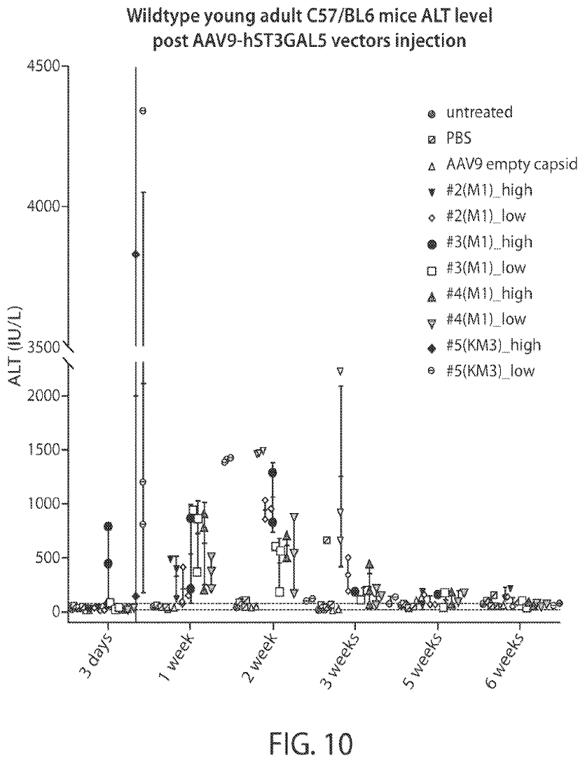

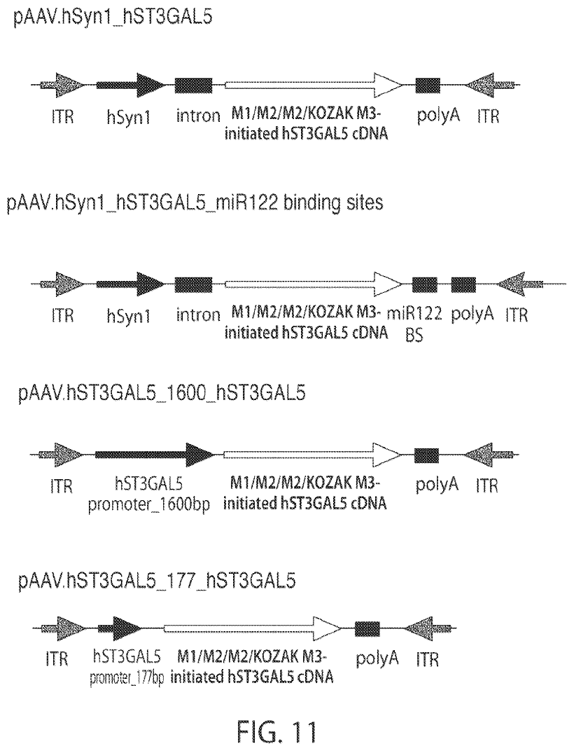

However, AAV9-hST3GAL5 vector at dosage 2×10¹⁴ vg/kg for neonates, and at 1×10¹⁴ vg/kg for young adults compromised survival (FIGS. 9A-9B), while a dosage at 2.7×10¹³ vg/kg were tolerable in wild type mice (FIG. 9B). In addition, after recording elevations of hepatic alanine aminotransferase following AAV9-hST3GAL5 injection (FIG. 10), four hST3GAL5-microRNA-122 (miR-122) binding sites constructs were also produced (FIG. 6) to attempted to de-target hST3GAL5 transgene expression from liver through endogenous miR-122 (FIG. 6). Studies were designed to elucidate pathomechanism and to optimize the vector design to mitigate vector associated toxicity (FIG. 11).

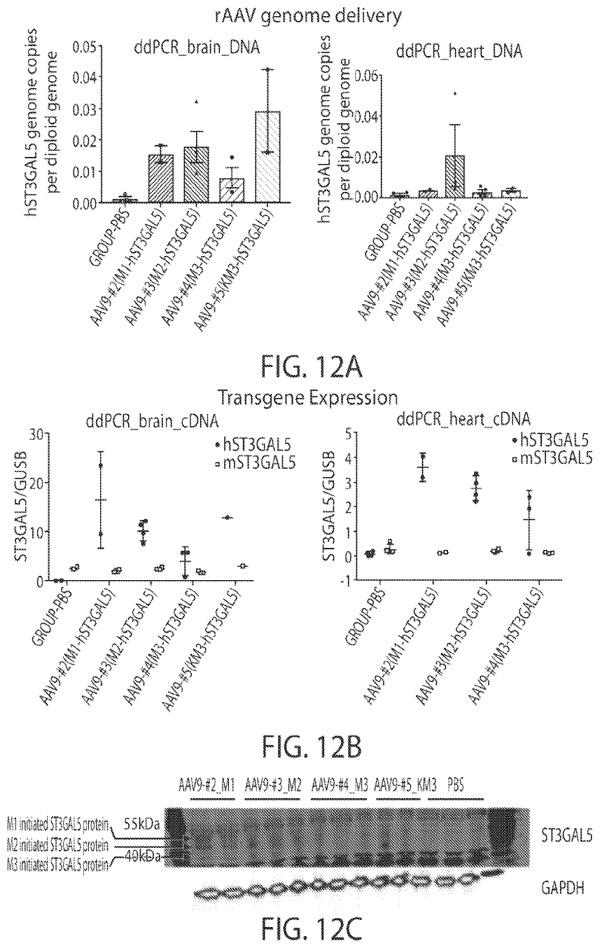

In parallel, neonatal wild type mice were injected with AAV9-ST3GAL5 protein isoforms via direct CNS injection. Enhanced rAAV genome biodistribution and hST3GAL5 over-expression was measured in the brain tissue by Droplet Digital PCR (ddPCR) and Western blot, respectively (FIGS. 12A-12B). No short-term vector-associated toxicity (FIGS. 12A-12B) was observed.

### Sequence Listing

The contents of the text file submitted electronically herewith are incorporated herein by reference in their entirety: A computer readable format copy of the Sequence Listing (filename: U0120.70113WO00-SEQ.txt, date recorded: Apr. 8, 2019, file size ~254,509 bytes).

The skilled artisan recognizes that certain sequences in the Sequence Listing are represented as linear nucleic acid sequences corresponding to circular plasmid sequences. Accordingly, in some embodiments, sequences described herein represent a contiguous polynucleoide (e.g., sequences sharing a continuous phosphate backbone), such that the first base and the last base of the linear representation are positioned next to one another. The Sequence listing contains the sequences as shown below:
construct #1_pAAVCBA opt_FL_h ST3GAL5-1a-2 (SEQ ID NO: 1)
construct #3_pAAVCBA Opt_M2_hST3GAL51a-2 (SEQ ID NO: 3)
construct #4_pAAVCBA Opt_M3_hST3GAL5-1a-2 (SEQ ID NO: 4)
construct #5_pAAVCBA_Opt_KM3_hST3GAL5-1a-2 (SEQ ID NO: 5)
construct #6_pAAVCBA FL_hST3GAL5-1a-2 (SEQ ID NO: 6)
construct #7_pAAVCBA FL_opt_M3_hST3GAL5-1a-2 (SEQ ID NO: 7)
Human GM3S Ia Type 2 isoform M1 variant amino acid sequence (SEQ ID NO: 8)
Human GM3S Ia Type 2 isoform M2 variant amino acid sequence (SEQ ID NO: 9)
Human GM3S Ia Type 2 isoform M3 variant amino acid sequence (SEQ ID NO: 10)
construct #2_miR122 BS pAAV CBA opt_M1_hST3GAL5-1a-2_mir122 binding (SEQ ID NO: 11)
construct #3_miR122 BS pAAV CBA opt_M2_hST3GAL5-1a-2_mir122 binding (SEQ ID NO: 12)
construct #4_miR122 BS pAAV CBA opt_M3_hST3GAL5-1a-2_mir122 binding (SEQ ID NO: 13)
construct #5_miR122 BS pAAV CBA opt_KM3_hST3GAL5-1a-2_mir122 binding (SEQ ID NO: 14)
pAAV_hSyn1_EGFP_miR122BS (SEQ ID NO: 15)
pAAV_hSyn1_intron_EGFP_miR122BS (SEQ ID NO: 16)
pAAV_hSyn1_intron_opt_KM3_hST3GAL5-1a-2_miR122BS (SEQ ID NO: 17)
pAAV_hSyn1_intron_opt_M1_hST3GAL5-1a-2_miR122BS (SEQ ID NO: 18)
pAAV_hSyn1_intron_opt_M2_hST3GAL5-1a-2_miR122BS (SEQ ID NO: 19)
pAAV_hSyn1_intron_opt_M3_hST3GAL5-1a-2_miR122BS (SEQ ID NO: 20)
pAAV_hSyn1_opt_KM3_hST3GAL5-1a-2 (SEQ ID NO: 21)
pAAV_hSyn1_opt_KM3_hST3GAL5-1a-2_miR122BS (SEQ ID NO: 22)
pAAV_hSyn1_opt_M1_hST3GAL5-1a-2 (SEQ ID NO: 23)
pAAV_hSyn1_opt_M1_hST3GAL5-1a-2_miR122BS (SEQ ID NO: 24)
pAAV_hSyn1_opt_M2_hST3GAL5-1a-2 (SEQ ID NO: 25)
pAAV_hSyn1_opt_M2_hST3GAL5-1a-2_miR122BS (SEQ ID NO: 26)
pAAV_hSyn1_opt_M3_hST3GAL5-1a-2 (SEQ ID NO: 27)
pAAV_hSyn1_opt_M3_hST3GAL5-1a-2_miR122BS (SEQ ID NO: 28)
pAAV_phST3GAL5_177_EGFP (SEQ ID NO: 29)
pAAV_phST3GAL5_177_KM3_hST3GAL5-1a-2 (SEQ ID NO: 30)
pAAV_phST3GAL5_177_M1_hST3GAL5-1a-2 (SEQ ID NO: 31)
pAAV_phST3GAL5_177_M2_hST3GAL5-1a-2 (SEQ ID NO: 32)
pAAV_phST3GAL5_177_M3_hST3GAL5-1a-2 (SEQ ID NO: 33)
pAAV_phST3GAL5_1600_EGFP (SEQ ID NO: 34)
pAAV_phST3GAL5_1600_KM3_hST3GAL5-1a-2 (SEQ ID NO: 35)
pAAV_phST3GAL5_1600_M1_hST3GAL5-1a-2 (SEQ ID NO: 36)
pAAV_phST3GAL5_1600_M2_hST3GAL5-1a-2 (SEQ ID NO: 37)
pAAV_phST3GAL5_1600_M3_hST3GAL5-1a-2 (SEQ ID NO: 38)
Wild-type Human GM3S Ia Type 2 isoform M1 variant coding sequence_codon un-optimized (SEQ ID NO: 39)
Human GM3S Ia Type 2 isoform M1 variant coding sequence_codon optimized (SEQ ID NO: 40)
Human GM3S Ia Type 2 isoform M2 variant coding sequence_codon optimized (SEQ ID NO: 41)
Human GM3S Ia Type 2 isoform M3 variant coding sequence_codon optimized (SEQ ID NO: 42)
Human GM3S Ia Type 2 isoform KM3 variant coding sequence_codon optimized (SEQ ID NO: 43)

1-41. (canceled)

42. A recombinant adeno-associated virus (rAAV) comprising:
(i) a capsid protein; and
(ii) an isolated nucleic acid of comprising:
(a) a first region comprising a first adeno-associated virus (AAV) inverted terminal repeat (ITR); and
(b) a second region comprising a transgene comprising a nucleic acid sequence that consists of the nucleotide sequence set forth in SEQ ID NO: 43 and encodes a monosialodihexosylganglioside synthase (GM3S) Ia Type 2 protein isoform.

43. The rAAV of claim 42, wherein the capsid protein is an AAV9 capsid protein or a variant thereof.

44. The rAAV of claim 43, wherein the AAV9 capsid protein variant is AAV.PHP.B capsid protein or AAB.PHP.eb.

45. The rAAV of claim 42, wherein the rAAV is formulated for delivery to the brain.

46. A composition comprising the rAAV of claim 42, and a pharmaceutical acceptable excipient.

47. A method for expressing one or more GM3S protein isoforms in a cell, the method comprising:
delivering to the cell the rAAV of claim 42.

48-56. (canceled)

57. The rAAV of claim 42, wherein the transgene further comprises a promoter operably linked to the nucleic acid sequence encoding the GM3 synthase Ia Type 2 protein isoform.

58. The rAAV of claim 57, wherein the promoter is a chicken beta-actin (CBA) promoter comprising a CMV enhancer sequence.

59. The rAAV of claim 42, further comprising a third region comprising a second adeno-associated virus (AAV) inverted terminal repeat (ITR), or a variant thereof, wherein the first region and the third region flank the transgene.

60. The rAAV of claim 59, wherein the first region and/or the third region is an AAV2 ITR or a variant thereof.

61. The rAAV of claim 42, further comprising a fourth region comprising one or more miRNA binding sites.

62. The rAAV of claim 61, wherein the one or more miRNA binding sites are miR-122 binding sites.


SEQUENCE LISTING (43 sequences)

SEQ ID NO: 1 - DNA, 3732 bp, synthetic construct
  misc_feature 1..3732: note = Synthetic Polynucleotide
  source 1..3732: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 3603 bp, synthetic construct
  misc_feature 1..3603: note = Synthetic Polynucleotide
  source 1..3603: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 3519 bp, synthetic construct
  misc_feature 1..3519: note = Synthetic Polynucleotide
  source 1..3519: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 3419 bp, synthetic construct
  misc_feature 1..3419: note = Synthetic Polynucleotide
  source 1..3419: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgacctggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 3441 bp, synthetic construct
  misc_feature 1..3441: note = Synthetic Polynucleotide
  source 1..3441: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 3732 bp, synthetic construct
  misc_feature 1..3732: note = Synthetic Polynucleotide
  source 1..3732: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 3732 bp, synthetic construct
  misc_feature 1..3732: note = Synthetic Polynucleotide
  source 1..3732: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccacgcgcccctcctccgcatctgagcgggggagcggcggcccccagctgaatgggcgcgagagcggcgctgggggcgggtgggggcgcggggtaccgggctggcggccggccggcgccccctcattagtatgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - protein, 418 aa, synthetic construct
  REGION 1..418: note = Synthetic Polypeptide
MRTKAAGCAERRPLQPRTEAAAAPAGRAMPSEYTYVKLRSDCSRPSLQWYTRAQSKMRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 9 - protein, 390 aa, synthetic construct
  REGION 1..390: note = Synthetic Polypeptide
MPSEYTYVKLRSDCSRPSLQWYTRAQSKMRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 10 - protein, 362 aa, synthetic construct
  REGION 1..362: note = Synthetic Polypeptide
MRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 11 - DNA, 6496 bp, synthetic construct
  misc_feature 1..6496: note = Synthetic Polynucleotide
  source 1..6496: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 12 - DNA, 6412 bp, synthetic construct
  misc_feature 1..6412: note = Synthetic Polynucleotide
  source 1..6412: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 13 - DNA, 6328 bp, synthetic construct
  misc_feature 1..6328: note = Synthetic Polynucleotide
  source 1..6328: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 14 - DNA, 6334 bp, synthetic construct
  misc_feature 1..6334: note = Synthetic Polynucleotide
  source 1..6334: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 15 - DNA, 4725 bp, synthetic construct
  misc_feature 1..4725: note = Synthetic Polynucleotide
  source 1..4725: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 16 - DNA, 4967 bp, synthetic construct
  misc_feature 1..4967: note = Synthetic Polynucleotide
  source 1..4967: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 17 - DNA, 5318 bp, synthetic construct
  misc_feature 1..5318: note = Synthetic Polynucleotide
  source 1..5318: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 18 - DNA, 5480 bp, synthetic construct
  misc_feature 1..5480: note = Synthetic Polynucleotide
  source 1..5480: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 19 - DNA, 5396 bp, synthetic construct
  misc_feature 1..5396: note = Synthetic Polynucleotide
  source 1..5396: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 20 - DNA, 5311 bp, synthetic construct
  misc_feature 1..5311: note = Synthetic Polynucleotide
  source 1..5311: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 21 - DNA, 5004 bp, synthetic construct
  misc_feature 1..5004: note = Synthetic Polynucleotide
  source 1..5004: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 22 - DNA, 5076 bp, synthetic construct
  misc_feature 1..5076: note = Synthetic Polynucleotide
  source 1..5076: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 23 - DNA, 5166 bp, synthetic construct
  misc_feature 1..5166: note = Synthetic Polynucleotide
  source 1..5166: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 24 - DNA, 5238 bp, synthetic construct
  misc_feature 1..5238: note = Synthetic Polynucleotide
  source 1..5238: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 25 - DNA, 5082 bp, synthetic construct
  misc_feature 1..5082: note = Synthetic Polynucleotide
  source 1..5082: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 26 - DNA, 5154 bp, synthetic construct
  misc_feature 1..5154: note = Synthetic Polynucleotide
  source 1..5154: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 27 - DNA, 4998 bp, synthetic construct
  misc_feature 1..4998: note = Synthetic Polynucleotide
  source 1..4998: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 28 - DNA, 5070 bp, synthetic construct
  misc_feature 1..5070: note = Synthetic Polynucleotide
  source 1..5070: mol_type = other DNA
tggtacccaaacaccattgtcacactccacaaacaccattgtcacactccacaaacaccattgtcacactccaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaattcatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 29 - DNA, 4372 bp, synthetic construct
  misc_feature 1..4372: note = Synthetic Polynucleotide
  source 1..4372: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 30 - DNA, 4729 bp, synthetic construct
  misc_feature 1..4729: note = Synthetic Polynucleotide
  source 1..4729: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 31 - DNA, 4891 bp, synthetic construct
  misc_feature 1..4891: note = Synthetic Polynucleotide
  source 1..4891: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 32 - DNA, 4807 bp, synthetic construct
  misc_feature 1..4807: note = Synthetic Polynucleotide
  source 1..4807: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 33 - DNA, 4723 bp, synthetic construct
  misc_feature 1..4723: note = Synthetic Polynucleotide
  source 1..4723: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 34 - DNA, 5796 bp, synthetic construct
  misc_feature 1..5796: note = Synthetic Polynucleotide
  source 1..5796: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcctactagacgcg

SEQ ID NO: 35 - DNA, 6153 bp, synthetic construct
  misc_feature 1..6153: note = Synthetic Polynucleotide
  source 1..6153: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtgccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 36 - DNA, 6315 bp, synthetic construct
  misc_feature 1..6315: note = Synthetic Polynucleotide
  source 1..6315: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 37 - DNA, 6231 bp, synthetic construct
  misc_feature 1..6231: note = Synthetic Polynucleotide
  source 1..6231: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 38 - DNA, 6147 bp, synthetic construct
  misc_feature 1..6147: note = Synthetic Polynucleotide
  source 1..6147: mol_type = other DNA
tggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtaacagacccagacttgagagagtccagatatcccgatatctggagaacaaaggcattcctaattttgctttaaaaataataatattgattattgcaaaatatagtaattagggaaaattaatcctttatcacaaacccttgtagcagaagacatctgcccatatatacaagcattacaagcattgtaccttagttggatatgtccctcctcttattttcaggaatgccctgctggagtgcagtggcgatctcggctcactgtagcctctgcctcccgggttcaagcgattctcctgcctcagccccgtgagtagctgagacaacaggcttgcgccaccacgcccggccaatatttgtatttctagtagagacggggttttgccatgttggccagctggtcttgaactcctgacatcaggtgatctgtccgtctcagcctcccaaagtgctgggattacgcgtgagccactgtgcccagccaccactttactttcttaataaacttgcttttactttgcactgtggacttactctgaattgtttcttgcgtgagatcgaagaaccctctcttggggtctagatcgggacccctttcctgtaacacatgggccttgggctgatctccaagattaagagggcaagatactcagctgcaagggtcagctgtcgctgaccccagcagctgctctgccttggccaacttaccggatttaaatcgcccagtgacagcttttcctccagtattttcaaaatgctcccaatatcctcacgttagaacaatccagaaatagtcttatggtaaaactcttttgggaaatgtgaaagtagagtcctcccaccttttcctttgcaacgcttttgccccaacctcgatcgcccaggcagcacattttttccggcacccccaccaagggaagaacagagacatctccaaaccagctgccctgcgccaagctgttacaaaatagagccacaagtctcccggtttatgcaaacatatttggctcggtgtctaggatcttctttttcaaactcagaaggagatcacaggcacacggccgcggtgctgtgtacgcccctctgaaaggaagttctgcaagtcgaccgacttcactggcttcactgaagtccggtccgaaaccaccggaaagttgcaggcgcacagtatacgttcagctgtgtccaagaaataacctatcaatcactgccttaagctttgtttccttcctgaattttcttattttcacgaatatttatgctttcacaaactgtatgtcatttcctctccagaaccaaggggggctcgccccagcctggcccccgccccgggtgcgtccctgcagggggcgccctgctctggcaatgcccaccgcaccgcagggctgactggccgcgcgcccgggggcgggggccgccgcggaggcatcgtgctctccgattggccgtccgcgtcctcgtgacgtcagaccccgcccacgcgcccctcctccgcatctgagcgggggagcggcggcccccagctgaatgggcgcgagagcggcgctgggggcgggtgggggcgcggggtaccgggctggcggccggccggcgccccctcattagtatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttgaacgcg

SEQ ID NO: 39 - DNA, 1257 bp, synthetic construct
  misc_feature 1..1257: note = Synthetic Polynucleotide
  source 1..1257: mol_type = other DNA
atgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttga

SEQ ID NO: 40 - DNA, 1257 bp, synthetic construct
  misc_feature 1..1257: note = Synthetic Polynucleotide
  source 1..1257: mol_type = other DNA
atgagaactaaagcagcaggttgtgcagagagaagaccactccagcctagaactgaagcagcagcagcaccagcaggtagagcaatgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 41 - DNA, 1173 bp, synthetic construct
  misc_feature 1..1173: note = Synthetic Polynucleotide
  source 1..1173: mol_type = other DNA
atgccatccgagtacacctacgtgaagctgcggtctgactgcagccgcccatccctgcagtggtacacaagagcccagagcaagatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 42 - DNA, 1089 bp, synthetic construct
  misc_feature 1..1089: note = Synthetic Polynucleotide
  source 1..1089: mol_type = other DNA
atgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga

SEQ ID NO: 43 - DNA, 1095 bp, synthetic construct
  misc_feature 1..1095: note = Synthetic Polynucleotide
  source 1..1095: mol_type = other DNA
gccaccatgaggagaccctccctgctgctgaaggatatcctgaagtgcaccctgctggtgtttggcgtgtggatcctgtacatcctgaagctgaactacaccacagaggagtgtgacatgaagaagatgcactacgtggaccctgatcacgtgaagagggcccagaagtacgctcagcaggtgctgcagaaggagtgtagaccaaagtttgccaagacaagcatggctctgctgttcgagcaccggtactccgtggatctgctgccttttgtgcagaaggccccaaaggactctgaggctgagagcaagtacgatccccctttcggctttcgcaagttcagctccaaggtgcagaccctgctggagctgctgccagagcacgacctgcccgagcacctgaaggctaagacatgcaggaggtgcgtggtcatcggatccggaggaatcctgcacggactggagctgggacacaccctgaaccagttcgacgtggtcatcaggctgaactccgccccagtggagggctactctgagcacgtgggaaacaagaccacaatcagaatgacataccccgagggagctcctctgtctgacctggagtactacagcaacgatctgttcgtggccgtgctgtttaagtctgtggacttcaactggctgcaggctatggtgaagaaggagaccctgcccttttgggtgcggctgttcttttggaagcaggtggccgagaagatcccactgcagcccaagcacttccgcatcctgaaccctgtgatcatcaaggagacagcttttgacatcctgcagtactccgagccccagtctaggttctggggcagagataagaacgtgcctaccatcggagtgatcgccgtggtgctggctacacacctgtgcgacgaggtgtctctggccggctttggatacgatctgaaccagcctaggaccccactgcactactttgacagccagtgtatggccgctatgaacttccagaccatgcacaacgtgaccacagagacaaagttcctgctgaagctggtgaaagagggggtggtgaaagacctgagcgggggtattgaccgagagttttga